(19) United States
(12) Patent Application Publication
(10) Pub. No.: US 2014/0276166 A1
(43) Pub. Date: Sep. 18, 2014

(54) METHOD OF MEASURING BIOIMPEDANCE

(71) Applicant: CardioLogic Innovations Ltd, Neve Ilan, IL (US)

(72) Inventors: Oren DRORI, Binyamina (IL); Shimon ARAD, Tel Aviv (IL); Haim KRIEF, Hadera (IL)

(73) Assignee: CardioLogic Innovations Ltd, Neve Ilan, IL (US)

(21) Appl. No.: 14/208,465

(22) Filed: Mar. 13, 2014

Related U.S. Application Data
(60) Provisional application No. 61/780,934, filed on Mar. 13, 2013.
(60) Provisional application No. 61/910,055, filed on Nov. 28, 2013.

(51) Int. Cl. A61B 5/053; A61B 5/08; A61B 5/00; G01R 27/02

(52) U.S. Cl. CPC A61B 5/0537; G01R 27/02; A61B 5/0531; A61B 5/0809; A61B 5/4878; A61B 5/0536; A61B 5/0535
USPC 600/529; 702/19; 600/547

(57) ABSTRACT

A method for measuring the impedance of a portion of a subject, by passing a known current provided by a current source unit between a first pair of electrodes contacting the skin surface of the subject. The measuring of a voltage with a voltage measuring instrument, between at least one second pair of electrodes contacting the skin surface of the subject when the current source unit is passing the known current through the first pair of electrodes; calculating the bio-impedance of the portion of the subject based on the known current and the calibrated voltage.

### FIELD OF THE INVENTION

The present disclosure relates to the field of instrumentation, as well as related methods, for monitoring and evaluating biophysical measurements in the body. In particular, the disclosure relates to the measurement of electrical impedance on the body of a subject.

### BACKGROUND OF THE INVENTION

Bioimpedance is the response of a living organism (or a portion thereof, such as a body part, organ, tissue, or the like) to an externally applied electric current. It is a measure of the opposition to the flow of that electric current through the tissues. The measurement of the bioimpedance (or bioelectrical impedance) has proved useful as a non-invasive method for measuring various parameters of the body.

However, bioimpedance measurements are subject to many confounding factors that create challenges in maintaining accuracy, standardization and repeatability of measurements across subjects and across time.

The voltage readout from a bioimpedance measuring device may be affected by multiple factors. The resistivity of the biological sample (e.g., whole subject, body part, organ or tissue) is a major factor, but so is the size of the biological sample. As such, there is a need to control for the natural variability of subject size as it is a source of inaccurate readings in bioimpedance devices.

The voltage readout from a bioimpedance measuring device may also be affected by the breathing cycle of the subject, especially if the device is measuring chest bioimpedance. Chest impedance can change substantially depending on the portion of the chest volume taken up by air, which has a much greater resistivity than the surrounding tissue. Therefore, there is a need for controlling for this source of variability.

### SUMMARY OF THE INVENTION

It is one object of the current invention to disclose a method for measuring the impedance of a portion of a subject:
a. passing a known current provided by a current source unit between a first pair of electrodes contacting the skin surface of the subject;
b. measuring a voltage [symbol] with a voltage measuring instrument, between at least one second pair of electrodes contacting the skin surface of the subject when the current source unit is passing the known current through the first pair of electrodes;
c. calculating the bio-impedance of the portion of the subject based on the known current and the calibrated voltage [symbol] ,
   wherein the calibrated voltage [symbol] is calculated according to the following calibration formula:

V_{c}=Vₘ=( [symbol] _{A}_{ₘ}[(J(□)]\vec{r}′(\vec{r}))α(r)d\vec{r}− [symbol] _{A}_{_{c}}[(J(□)]\vec{r}′(\vec{r}))α(r)d\vec{ [symbol]})
wherein Aₘ is the volume of the portion of the subject enclosed by the second pair of electrodes; A_{c} is the volume of a standard portion of a measured subject enclosed by the second pair of electrodes; α(r) is a function containing the resistivity of a body according to the radius the cross section of the subject; and J(\vec{r}(\vec{r})) is the Jacobian of coordinate transformation from Cartesian coordinates \vec{r}′ to elliptic coordinates \vec{r}:

$$I ( \vec{r} ( \vec{r} ) ) = Det \vert \frac{d \vec{r} ( \vec{r} )}{d \vec{r}} \vert$$

further wherein the α(r) is calculated according to a solution of the poission equation:

$$∇ \cdot ( \frac{1}{ρ} ∇ ϕ ) = - {l}_{γ} ;$$

the φ is the electric potential according as a function of position on the thorax of the subject; the ρ is the impedance as a function of position in the thorax; and l_{γ} is zero except on the surface of the thorax.

It is another object of the present invention to provide the method as described above, wherein the cross section is an elliptic cross-section.

It is another object of the present invention to provide the method as described above, wherein said known current is an alternating current having a frequency of 20 kHz or less.

It is another object of the present invention to provide the method as described above, wherein said known current is an alternating current having a frequency of 40 kHz or less.

It is another object of the present invention to provide the method as described above, wherein said known current is an alternating current having a frequency of 60 kHz or less.

It is another object of the present invention to provide the method as described above, wherein said bio-impedance is further used to determine thoracic fluid content.

It is another object of the present invention to provide the method as described above, wherein said step of calculating said impedance comprises substep of: calculating the bio-impedance of said portion of said subject based on said calibrated voltage v_{c} and said known current.

It is another object of the present invention to provide the method as described above, wherein when a(r) is a constant function said calibration formula is the following linear formula: v_{c}=vₘ−B(Pₘ−P_{c}), where B is a constant, Pₘ is the measured cross-section, and P_{c} is the standard cross-section size.

It is another object of the present invention to provide the method as described above, wherein B is deduced by linear regression based on an empiric measurement of thorax width and cross thorax impedance.

It is another object of the present invention to provide the method as described above, wherein said step of calculating the bio-impedance comprises substeps of:
calibrating said measured voltage vₘ with respect to the breathing cycle of said subject to provide a calibrated voltage v_{c}; and
calculating the bio-impedance of said portion of said subject based on said calibrated voltage v_{c}, and said known current.

It is another object of the present invention to provide the method as described above, wherein said step of breathing cycle calibration is performed according to an algorithm comprising further steps of:
taking a first plurality of voltage measurement over a period of time encompassing in aggregate at least two exhalation events;
from said first plurality of voltage measurements, selecting a second plurality of voltage measurements at or near the voltage troughs;
averaging said second plurality of voltage measurements.

It is another object of the present invention to provide the method as described above, wherein said vₘ is calibrated in respect to skin potential that should have been derived using either equi-spacing position, non-equi-spacing; and any combination thereof.

It is another object of the current invention to disclose a device configured to measure the impedance of a portion of a subject, comprising:
a current source unit capable of passing a known current through the subject's chest through a first pair of electrodes;
a voltage measuring unit capable of measuring a voltage between at least one second pair of electrodes when the current source unit is passing the known current through the portion of the subject through the first pair of electrodes, and when the first and the second pair of electrodes are placed on the subject; and
a controller, comprising at least one processor, configured to determine the impedance of the portion of the subject based on the known current and calibrated voltage [symbol] value based upon measured voltage; the calibrated voltage [symbol] is calculated by the formula:

V_{c}=Vₘ−( [symbol] _{A}_{ₘ}[(J(□)]\vec{r}′(\vec{r}))α(r)d\vec{r}− [symbol] _{A}_{_{c}}[(J(□)]\vec{r}′(\vec{r}))α(r)d\vec{ [symbol]})
where Aₘ is the volume of a portion of the subject enclosed by the second pair of electrodes; A_{c} is the volume of a standard portion of a measured subject enclosed by the second pair of electrodes; α(r) is a function containing the resistivity of a body according to the radius of a cross section of the subject; and K(\vec{r}(\vec{r})) is the Jacobian of coordinate transformation from Cartesian coordinates \vec{r}′ to elliptic coordinates

$$I ( \vec{r} ( \vec{r} ) ) = Det \vert \frac{d \vec{r} ( \vec{r} )}{d \vec{r}} \vert$$

further wherein the α(r) is calculated according to a solution of the poission equation:

$$∇ \cdot ( \frac{1}{ρ} ∇ ϕ ) = - {l}_{γ} ;$$

the φ is the electric potential according as a function of position on the thorax of the subject; the ρ is the impedance as a function of position in the thorax; and l_{γ} is zero except on the surface of the thorax.

In some embodiments of the current invention, the device as described above, wherein the cross section is an elliptic cross section.

It is according to a first aspect of the disclosure to introduce a device configured to measure the impedance of a portion of a subject. The device may comprise: a current source unit capable of passing a known current through the subject's chest through a first pair of the electrodes; a voltage measuring unit capable of measuring a voltage between a at least one further pair of the electrodes when the current source unit is passing the known current through the portion of the subject through the first pair of electrodes, and when the first and second pair of electrodes are placed on the subject; and a controller, comprising at least one processor, configured to determine the impedance of the portion of the subject based on the known current and calibrated voltage value based upon the measured voltage.

Variously, the known current may be an alternating current having a frequency of 20 kHz or less. Alternatively, the known current may be an alternating current having a frequency of 40 kHz or less. Alternatively again, the known current may be an alternating current having a frequency of 60 kHz or less.

Where appropriate, the controller may be further configured to calibrate the measured voltage Vm with respect to the size of the subject. Optionally, the size of the subject may be the size of the cross-section of the part of the body defined by the location of the electrodes.

Notably, the relationship between a measured cross-sectional size Pm and the voltage measure from a subject Vm may be linear. Accordingly, the calibrated voltage Vc may be determined according to the formula Vc=Vm−a(Pm−Pc), wherein the term a is a constant and the term Pc is a standard cross-section size.

According to another embodiment, B is deduced by linear regression based on an empiric measurement of thorax width and cross thorax impedance.

Optionally, the cross-section size may be a parameter selected from the group consisting of a circumference, a perimeter length, a thickness, a diameter, a radius, an axis length, a volume, a surface area and a cross-sectional area. Optionally, the cross-section size may be a perimeter length or a thickness.

It is further noted that the controller may be configured to calibrate the measured voltage with respect to the breathing cycle of the subject. Variously, the breathing cycle calibration may be performed according to an algorithm comprising the steps of: taking a plurality of voltage measurements over a period of time encompassing in aggregate at least one full inhalation/exhalation cycle; averaging the plurality of voltage measurements. Optionally, the breathing cycle calibration may be performed according to an algorithm comprising the steps of: taking a first plurality of voltage measurements over a period of time encompassing in aggregate at least two exhalation events; from the first plurality of voltage measurements, selecting a second plurality of voltage measurements at or near the voltage troughs; averaging the second plurality of voltage measurements.

The device may further comprise a fixed resistive element having a resistance R connectable to the current source unit and the voltage measuring unit, wherein the controller is configured to calculate a system impedance SI based on the voltage measured during the injection of a known current through the fixed resistive element, as well as to calibrate the measured bioimpedance BIM with respect to the system impedance SI to obtain a calibrated bioimpedance BIC Optionally, the calibrated bioimpedance BIC is calculated according to the formula: BIC=(BIM/SI)R.

Variously, the device may be configured to perform at least one process selected from the group consisting of plethysmograpy, impedance cardiography, pneumography, organ volumetry, tissue volumetry, tissue characterization, edema detection, ischemia detection, graft viability monitoring and graft rejection monitoring. For example, the tissue characterization may be cancer detection.

The device may be configured to measure impedance in the chest of the subject. Such a device may be incorporated into an electrical impedance tomography (EIT) system. Optionally, the EIT may be parametric EIT. Accordingly, the system may be configured to measure the level of pulmonary edema in at least one lung of the subject.

It is according to another aspect of the disclosure to teach a method for measuring the impedance of a portion of a subject, comprising the steps of: passing a known current provided by a current source unit between a first pair of electrodes contacting the skin surface of the subject; measuring a voltage, with a voltage measuring instrument, between at least one further pair of the electrodes contacting the skin surface of the subject when the current source unit is passing the known current through the first pair of electrodes; and calculating the bioimpedance of the portion of the subject based on the known current and a calibrated voltage value based upon the measured voltage. Variously, the known current may be an alternating current having a frequency of 20 kHz or less. Alternatively, the known current may be an alternating current having a frequency of 40 kHz or less. Alternatively again, the known current may be an alternating current having a frequency of 60 kHz or less. Optionally, the known current is an alternating current having a frequency of 100 kHz or less, and wherein the bioimpedance is further used to determine thoracic fluid content.

Where appropriate, the step of calculating the impedance comprises the substeps of: calibrating the measured voltage Vm with respect to the size of the subject to provide a calibrated voltage Vc; and calculating the bioimpedance of the portion of the subject based on the calibrated voltage Vc and the known current. Optionally, the size of the subject is the size of the cross-section of the part of the body defined by the location of the electrodes. Notably, the relationship between a measured cross-sectional size Pm and the voltage measured from a subject Vm may be linear. Accordingly, the calibrated voltage Vc may be determined according to the formula Vc=Vm−a(Pm−Pc), wherein the term a is a constant and the term Pc is a standard cross-section size.

Optionally, the cross-section size may be a parameter selected from the group consisting of a circumference, a perimeter length, a thickness, a diameter, a radius, an axis length, a volume, a surface area and a cross-sectional area. The cross-section size may be a perimeter length or a thickness.

Optionally, the step of calculating the bioimpedance may comprise the substeps of: calibrating the measured voltage Vm with respect the breathing cycle of the subject to provide a calibrated voltage Vc; and calculating the bioimpedance of the portion of the subject based on the calibrated voltage Vc and the known current.

Additionally, or alternatively, the substep of breathing cycle calibration may be performed according to an algorithm comprising the further substeps of: taking a plurality of voltage measurements over a period of time encompassing in aggregate at least one full inhalation/exhalation cycle; and averaging the plurality of voltage measurements.

Optionally, the substep of breathing cycle calibration is performed according to an algorithm comprising the further substeps of: taking a first plurality of voltage measurements over a period of time encompassing in aggregate at least two exhalation events; from the first plurality of voltage measurements, selecting a second plurality of voltage measurements at or near the voltage troughs; and averaging the second plurality of voltage measurements.

Another aspect of the disclosure is to disclose a method for measuring the impedance of a portion of a subject, comprising the steps of: passing a known current provided by a current source unit between a first pair of electrodes contacting the skin surface of the subject; measuring a first voltage, with a voltage measuring instrument, between at least one further pair of the electrodes contacting the skin surface of the subject when the current source unit is passing the known current through the first pair of electrodes; calculating the measured bioimpedance BIM of the portion of the subject based on the first voltage and the known current; passing the known current provided by the current source unit through a fixed resistive element having a resistance R; measuring a second voltage, with the voltage measuring instrument, when the current source unit is passing the known current through the fixed resistive element; calculate a system impedance SI based on the second voltage and the known current; and calibrating the measured bioimpedance BIM with respect to the system impedance SI to derive the calibrated bioimpedance BIC. Optionally, calibrated bioimpedance BIC is calculated according to the formula: BIC =(BIM/SI)R.

### BRIEF DESCRIPTION OF THE FIGURES

For a better understanding of the embodiments and to show how they may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1A is a schematic illustration showing a bioimpedance measurement device;

FIG. 1B is a schematic illustration showing a bioimpedance measurement device with amultiplexer;

FIG. 1C is a schematic illustration showing a bioimpedance measurement device with the electrodes being incorporated into a body contacting device;

FIG. 1D is a schematic illustration showing a bioimpedance measurement device with the electrodes being incorporated into multiple body contacting devices.

FIG. 2 shows a flow diagram for a method of measuring the bioimpedance of a portion of a subject.

FIG. 3 shows a flow diagram for the method of measuring the bioimpedance of a portion of a subject, which further includes a calibration step for calibrating the measured voltage with respect to the size of the subject.

FIGS. 4A-C show a flow diagram, with variations, for the method of measuring the bioimpedance of a portion of a subject, which further includes a calibration step for calibrating the measured voltage with respect to the breathing cycle.

FIG. 5A is a schematic illustration showing a bioimpedance measurement device with a fixed resistive element.

FIG. 5B is a schematic illustration showing a bioimpedance measurement device with a multiplexer and a fixed resistive element.

FIG. 6 shows a flow diagram for the method of measuring the bioimpedance of a portion of a subject, which further includes a calibration step for calibrating the measured voltage with respect to system impedance.

FIG. 7A shows a two-dimensional thorax model based on an axial CT image and employed for simulations, the image being segmented into 4 tissue types: heart, lung, other soft tissue and bone.

FIG. 7B shows the two-dimensional thorax model sampled into a lower resolution of 20×20 pixels, with a spatial resolution ranging between Δh=1 to 2 cm.

FIG. 8 shows the lower resolution two-dimensional thorax model of FIG. 7B, indicating the location of the electrodes used in the simulation.

FIG. 9A is a graph showing the normalized voltage values measured from simulated chests of a range of perimeter lengths (with the voltage measured from a chest with a perimeter length of 100 cm being 1) plotted against the corresponding chest perimeter length, with the lung resistivity being set at 500 Ωcm.

FIG. 9B is a graph showing the normalized voltage values measured from simulated chests of a range of perimeter lengths (with the voltage measured from a chest with a perimeter length of 100 cm being 1) plotted against the corresponding chest perimeter length, with the lung resistivity being set at 1000 Ωcm.

FIG. 9C is a graph showing the normalized voltage values measured from simulated chests of a range of perimeter lengths (with the voltage measured from a chest with a perimeter length of 100 cm being 1) plotted against the corresponding chest perimeter length, with the lung resistivity being set at 1500 Ωcm.

FIG. 10A is a graph showing the normalized voltage values measured from simulated chests of a range of perimeter lengths (with the voltage measured from a chest with a perimeter length of 100 cm being 1) plotted against the corresponding chest perimeter length, with the left lung resistivity being set at 500 Ωcm and the right lung resistivity being set at 1200 Ωcm.

FIG. 10B is a graph showing the normalized voltage values measured from simulated chests of a range of perimeter lengths (with the voltage measured from a chest with a perimeter length of 100 cm being 1) plotted against the corresponding chest perimeter length, with the left lung resistivity being set at 1200 Ωcm and the right lung resistivity being set at 500 Ωcm.

FIGS. 11A-B are graphs showing the voltage measurements taken during continuous (fast sampling) bioimpedance monitoring for over 10 seconds during tidal volume breathing (normal breathing).

FIG. 11C is a graph showing the voltage measurements taken during continuous (fast sampling) bioimpedance monitoring for over 10 seconds during deep breathing.

FIG. 11D is a graph showing the voltage measurements taken during continuous (fast sampling) bioimpedance monitoring for over 10 seconds while holding the breath.

FIG. 11E is a graph showing the voltage measurements taken during continuous (fast sampling) bioimpedance monitoring for over 10 seconds during maximum exhalation followed by holding the breath.

FIG. 12a-12d shows options for placing electrodes according to the equi-space method.

### DETAILED DESCRIPTION OF THE INVENTION

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figure and described in the specification, but only as indicated in the accompanying claims, with the proper scope determined only by the broadest interpretation of said claims.

Definitions

As used herein, the term “Electrical impedance” (also known as “impedance”) is referred hereinafter as the measure of the opposition that a circuit presents to the passage of a current when a voltage is applied. In quantitative terms, it is the complex ratio of the voltage to the current in an alternating current (AC) circuit. Complex impedance Z may be represented as a magnitude |Z| and phase shift (θ) in polar form, or a real part R and an imaginary part jX in rectangular form.

The term “Bioimpedance” is referred hereinafter as the electrical impedance of biological samples, such as whole body, body part, tissue, organ, cell and the like.

The term “Electrical resistivity” (also known as “resistivity”, “specific electrical resistance”, or “volume resistivity”) is referred hereinafter as an element of impedance (including bioimpedance) that quantifies how strongly a given material opposes the flow of electric current. Unlike resistance, resistivity is a characteristic property of a material and independent of size or shape. Resistivity is commonly represented as the Greek letter rho (ρ). The SI unit of electrical resistivity is the ohm meter (Ωm) although other units like the ohm centimeter (Ωcm) are also in use.

The term “F-EIT” referred hereinafter to Functional-EIT. F-EIT determines relative resistivity changes of each pixel during the measurement session that may be caused for example by ventilation or changes during breathing (vs. a baseline level, which is typically defined by the intra-thoracic impedance distribution at the end of expiration) and so identify a function such as breathing functionality—in oppose to absolute EIT (a-EIT) which determines the absolute state of the lungs and other organs allowing to determine directly pathophysiological state directly.

The present invention provides means and method for enhanced bioimpedance, in which the measurements are calibrated by means of either one of (a delivery of low frequency alternating current; (b) body dimension calibration (as described herein below); (c) breathing cycle calibration (as described herein below); and/or (d) fixed resistive element calibration (as described herein below) and any combination thereof.

Before describing the enhanced calibration, the following provides description of the Bioimpedance measuring device.

Bioimpedance Measuring Device

Reference is now made to FIG. 1A, which is a schematic diagram of a bioimpedance measuring device 100. The device 100 includes at least two electrodes 110 for delivering an electric current, attachable to the skin surface of a subject 10 and connected to a current source unit 230. The device 100 also includes at least two further electrodes 120 for measuring voltage, attachable to the skin surface of a subject 10 and connected to a voltage measuring unit 240.

The current source unit 230 may be an alternating current AC source capable of delivering a known alternating current in one or more defined amplitudes and frequencies through the electrodes 110 connected thereto.

The voltage measuring unit 240 may be configured to measure the voltage between the electrodes 120 connected thereto.

The current source unit 230 and the voltage measuring unit 240 may be incorporated in a controller 225. The controller 225 may further include one or more processors 250. The processor(s) may control current source 230 and the voltage measuring unit 240 such that when the current source unit 230 is delivering the known current through a portion of the subject through the first pair of electrodes 110, the voltage measuring instrument 240 is measuring the voltage between said at least one further pair of the electrodes 120. The processor(s) may further control the current source 230 and the voltage measuring unit 240 such that they are active only when the first and second pair of electrodes 110, 120 are placed on the subject. The controller 225 may further include one or more processors configured to determine the impedance based on the known current delivered by the current source unit 230 and the voltage measured by the voltage measurement instrument 240.

The processor(s) 250 may further be configured to calculate a variety of parameters related to or derived from the impedance. These parameters may include the real component of the impedance, the imaginary component of the impedance, the magnitude (|Z|) of the impedance, the phase shift (θ) of the impedance, resistivity (ρ) and the like. Further, the voltage readout from a bioimpedance measuring device may be affected by multiple factors that may be a source of noise or inaccuracies. As such, the processor(s) 250 may be configured to calibrate or normalize the measured data in various ways, some of which are detailed below.

The microprocessor(s) 250 (or one or more other microprocessors) may be configured to record and analyze the voltage changes measured in the electrodes connected to the voltage measurement unit 240. Optionally, the data analysis and the image generation may be executed in a separate data analysis unit, e.g., a computer that is connected (via wire or wirelessly) to the controller.

In various embodiments of the disclosure, the processor may be a computing platform or distributed computing system for executing a plurality of instructions. Optionally, the processor includes or accesses a volatile memory for storing instructions, data or the like. Additionally or alternatively, the data processor may access a non-volatile storage, for example, a magnetic hard disk, flash-drive, removable media or the like, for storing instructions and/or data. Optionally, a network connection may additionally or alternatively be provided. User interface devices may be provided such as visual displays, audio output devices, tactile outputs and the like. Furthermore, as required, user input devices may be provided such as keyboards, cameras, microphones, accelerometers, motion detectors or pointing devices such as mice, roller balls, touch pads, touch sensitive screens or the like.

In various embodiments of the disclosure, conducting electrodes are attached to the skin of the subject and small alternating currents are applied to some or all of the electrodes. In other embodiments, the electrodes can be implanted. A non-limiting example of the use of implanted electrodes is in an AICD (Active Implantable Cardiac Device) such as a pacemaker, CRT, CRT-D or ICD. For these, some of the electrodes are subcutaneous, on the device's case, and some are implanted leads.

With reference to FIG. 1B, the controller 225 may include at least one multiplexer 260. The connection of each electrode 130 with the current source unit 230 or the voltage measurement unit 240 may be controlled by the multiplexer 260, such that each electrode is capable of being a part of the electrode pair injecting current to the skin surface subject 10, or to be a part of the electrode pair measuring voltage changes.

As mentioned above, in other embodiments, the electrodes can be implanted, such as in AICD.

With reference to FIG. 1C, the electrodes 130 may be incorporated into a body contacting device 300, which is configured to facilitate the contacting of the electrode with the skin surface. Alternatively, with reference to FIG. 1D, a first subset of electrodes 130 may be incorporated in a first body contacting device 300 and a second subject of electrodes 130 may be incorporated in a second body contacting device 300′. It will be appreciated that the electrodes 130 may be subdivided into three, four, five or more separate body contacting devices 300. The bioimpedance measuring device 100 may be a device configured to perform plethysmograpy, impedance cardiography (ICG), pneumography, organ volumetry, tissue volumetry, tissue characterization, edema detection, ischemia detection, graft viability monitoring or graft rejection monitoring. The tissue characterization may be cancer detection.

The bioimpedance measuring device 100 may be an electrical impedance tomography (EIT) device, an electrocardiography (ECG) device, a body surface mapping device, and the like. The EIT may be parametric EIT (pEIT). The bioimpedance measuring device 100 may be configured to perform bioimpedance analysis (BIA) with the injection of an alternating current at one defined frequency, or bioimpedance spectroscopy (BIS) with the injection of an alternating current at more than one defined frequencies.

The EIT or pEIT may be for the purpose of monitoring the level of fluid, e.g., extracellular fluid, in one or more organs of the chest cavity in a subject. The organ may be a lung. The chest bioimpedance image may be for the purpose of monitoring pulmonary edema, which is characterized by a buildup of extracellular fluid in the lungs. The pulmonary edema may be cardiogenic, caused by improper heart function, e.g., congestive heart failure (CHF).

Alternatively, the pulmonary edema may be non-cardiogenic and caused by, e.g., an injury to one or both of the lungs.

Accordingly, with reference to FIG. 2, the present disclosure describes a method for measuring the bioimpedance of a portion of a subject, comprising the steps of:
passing a known current provided by a current source unit between a first pair of electrodes contacting the skin surface of the subject (402);
measuring a voltage, with a voltage measuring instrument, between at least one other pair of the electrodes contacting the skin surface of the subject when the current source unit is passing the known current through the first pair of electrodes (404); and
calculating the bioimpedance of the portion of the subject based on the measured voltage and the known current (406).

It is noted that in order to implement the methods, devices or systems of the disclosure, various tasks may be performed or completed manually, automatically, or combinations thereof. Moreover, according to selected instrumentation and equipment of particular embodiments of the methods or systems of the disclosure, some tasks may be implemented by hardware, software, firmware or combinations thereof using an operating system. For example, hardware may be implemented as a chip or a circuit such as an ASIC, integrated circuit or the like. As software, selected tasks according to embodiments of the disclosure may be implemented as a plurality of software instructions being executed by a computing device using any suitable operating system.

Calibration According to Cross Section of a Subject and Body Dimension

It is one object of the current invention to disclose a method for measuring the impedance of a portion of a subject:
a. passing a known current provided by a current source unit between a first pair of electrodes contacting the skin surface of the subject;
b. measuring a voltage [symbol] with a voltage measuring instrument, between at least one second pair of electrodes contacting the skin surface of the subject when the current source unit is passing the known current through the first pair of electrodes;
c. calculating the bio-impedance of the portion of the subject based on the known current and the calibrated voltage [symbol] ,
   wherein the calibrated voltage [symbol] is calculated according to the following calibration formula:

V_{c}=Vₘ−( [symbol] _{A}_{ₘ}[(J(□)]\vec{r}′(\vec{r}))α(r)d\vec{r}− [symbol] _{A}_{_{c}}[(J(□)]\vec{r}′(\vec{r}))α(r)d\vec{ [symbol]})
where Aₘ is the volume of the portion of the subject enclosed by the second pair of electrodes; A_{c} is the volume of a standard portion of a measured subject enclosed by the second pair of electrodes; α(r) is a function containing the resistivity of a (body according to the radius the cross section of the subject; and J(\vec{r}(\vec{r})) is the Jacobian of coordinate transformation from Cartesian coordinates \vec{r}′ to elliptic coordinates \vec{r}:

$$I ( \vec{r} ( \vec{r} ) ) = Det \vert \frac{d \vec{r} ( \vec{r} )}{d \vec{r}} \vert$$

further wherein the α(r) is calculated according to a solution of the poission equation:

$$∇ \cdot ( \frac{1}{ρ} ∇ ϕ ) = - {l}_{γ} ;$$

the φ is the electric potential according as a function of position on the thorax of the subject; the ρ is the impedance as a function of position in the thorax; and is zero except on the surface of the thorax.

it is another object of the current invention to provide the method as described above, wherein the cross section is an elliptic cross-section.

It is another object of the current invention to disclose a device configured to measure the impedance of a portion of a subject, comprising:
a current source unit capable of passing a known current through the subject's chest through a first pair of electrodes;
a voltage measuring unit capable of measuring a voltage between at least one second pair of electrodes when the current source unit is passing the known current through the portion of the subject through the first pair of electrodes, and when the first and the second pair of electrodes are placed on the subject; and
a controller, comprising at least one processor, configured to determine the impedance of the portion of the subject based on the known current and calibrated voltage [symbol] value based upon measured voltage; the calibrated voltage [symbol] is calculated by the formula:

V_{c}=Vₘ−( [symbol] _{A}_{ₘ}[(J(□)](\vec{r}))α(r)d\vec{r}− [symbol] _{A}_{_{c}}[(J(□)]\vec{r}′(\vec{r}))α(r)d\vec{ [symbol]})
where Aₘ is the volume of a portion of the subject enclosed by the second pair of electrodes; A_{c} is the volume of a standard portion of a measured subject enclosed by the second pair of electrodes; α(r) is a function containing the resistivity of a body according to the radius of a cross section of the subject; and J(\vec{r}(\vec{r})) is the Jacobian of coordinate transformation from Cartesian coordinates \vec{r}′ to elliptic coordinates \vec{r}:

$$J ( {{r}^{′}}^{} ( {r}^{->} ) ) = Det \vert \frac{d {{r}^{′}}^{} ( {r}^{->} )}{d {r}^{->}} \vert$$

further wherein the α(r) is calculated according to a solution of the poission equation:

$$v \cdot ( \frac{1}{ρ} {v}_{ϕ} ) = - {l}_{y} ;$$

the φ is the electric potential according as a function of position on the thorax of the subject; the ρ is the impedance as a function of position in the thorax; and l_{γ} is zero except on the surface of the thorax.

In some embodiments of the current invention, the device as described above, wherein the cross section is an elliptic cross section.

It should be emphasized that the number n of electrode in the system can be any number of electrodes. And the calibration can be done to a portion of the electrode or the entire system. For example, in a given system of n electrode, one can calibrate only y electrode. The y electrode is basically a sub-system of the n electrode system. In such a case, the calibration is performed only to the y electrode out of the n electrode system.

In other words, the y electrodes, can be a sub-system of n>4 electrodes.

The calculation of the calibration is done by determining the jacobian of the coordinates. In differential calculus, the Jacobian matrix is a matrix of partial derivatives of transforming from one set of coordinates to another.

Performing the calibration may be done by placing the electrodes on an elliptic shaped organ. For example, the chest, the arm, the hip etc. After placing the electrodes around such organ (or for example, just two electrodes at antipodes points) a slicing of the organ is performed producing a surface with elliptic symmetry. In this elliptically symmetric slice, it is easier to perform any calculation regarding the calibration by transforming the regular Cartesian coordinates to elliptic coordinates. After producing the Jacobian matrix:

$$\frac{d {{r}^{′}}^{} ( {r}^{->} )}{d {r}^{->}}$$

Using the determinant of this matrix

$$Det \vert \frac{d {{r}^{′}}^{} ( {r}^{->} )}{d {r}^{->}} \vert$$

will give the transformation factor in order to calculate the calibration in elliptic coordinates.

The following provides a simplified linear correlation for the Body dimension calibration

The various options described for the bioimpedance measuring device 100 and its components as described with reference to FIGS. 1A-D above are also options for the body dimension calibration described below. Further, the devices and methods relating to body dimension calibration may be used in combination with the delivery of low frequency alternating current (as described above), breathing cycle calibration (as described below) and/or fixed resistive element calibration (as described below).

The voltage readout from a bioimpedance measuring device may be affected by multiple factors. The resistivity of the biological sample (e.g., whole subject, body part, organ or tissue) is a major factor, but so is the size of the biological sample. As such, the natural variability of subject size is a source of inaccurate readings in bioimpedance devices.

The bioimpedance measuring device of the disclosure, or the processor(s) therein involved in analyzing the measurements, may be configured to calibrate the voltage readout to subject size. More specifically, the device may be configured to calibrate the voltage readout based on the size of the cross-section of the part of the body where the electrodes are placed. For example, if the electrodes are placed around the arm, the voltage readout is calibrated to the size of the cross-section of the arm as defined by the electrodes. Similarly, if the electrodes are placed around the chest, the voltage readout is calibrated to the size of the cross-section of the chest as defined by the electrodes. It will be appreciated that such a calibration maybe be done for other body parts, such as the legs, the neck, the head, and the like. The size of the body portion may be the circumference, the perimeter length, a thickness, the diameter, the radius, an axis length (e.g. cross sectional width or length), the volume, the surface area, the cross-sectional area. In a particular embodiment of the disclosure, the size may be the circumference, the perimeter length or the like. In a particular embodiment of the disclosure, the size may be the thickness, the diameter, an axis length (e.g., cross sectional width or length), or the like.

The relationship between the voltage readout and subject size may be linear. For example, as demonstrated in Example 2, the relationship between the voltage readout and chest perimeter may be linear. That is, the relationship between voltage and chest perimeter may be described by the formula:

V=aP+b, where V is voltage, P is a cross-sectional size of the chest (which may be, but not limited to, a chest perimeter length) and the terms a and b are constants. Thus, by calibrating the measured voltage readout to a theoretical voltage readout based on subjects having a standard cross sectional size (e.g., chest perimeter length), it is possible to eliminate the differences in the voltage readout that arises from differences in the size of the patient, and arrive at a normalized voltage readout and calculated resistance that better reflects biophysical differences independent from body dimension, such as thoracic of lung fluid content.

Based on the linear relationship, the measured voltage Vm from a subject with a measured chest cross-sectional size Pm may be described by the formula:

Vm=aPm+b

Similarly, a calibrated voltage Vc, based on a theoretical subject with all biophysical conditions identical except for the chest cross-sectional size Pc (Pc being a predetermined value, e.g., 100 cm), may be described by the formula:

Vc=aPc+b

As such, the difference between the measured voltage Vm and the calibrated voltage Vc may be expressed in the following manner:

Vm−Vc=aPm +b−aPc−b,

Which, following the constant b being eliminated, is equivalent to:

Vm−Vc=aPm−aPc=a(Pm−Pc)

Thus, following the acquisition of the measured voltage Vm and the measured chest perimeter Pm, the calibrated voltage Vc may be calculated according to the formula:

Vc=Vm−a(Pm−Pc), where the terms a and Pc are constants. As such, actual voltage Vm measured by the electrodes connected to the voltage measuring unit may be converted to a calibrated voltage Vc, with the calibrated voltage Vc then being used for the subsequent analysis, such as the determination of impedance or resistivity.

It will be appreciated that the linear relationship between voltage readout and chest size is not limited to perimeter length. Other chest parameters, such as the thickness, the diameter, or an axis length such as cross-sectional width or cross-sectional length may have a linear relationship with the voltage readout. As such, the term P (i.e., the measured Pm and standard Pc) may alternatively refer to perimeter length, thickness, diameter, or an axis length such as crosssectional width or cross-sectional length.

Accordingly, with reference to FIG. 3, the present disclosure describes a method for measuring the impedance of a portion of a subject, comprising the steps of:
passing a known current provided by a current source unit between a first pair of electrodes contacting the skin surface of the subject (402);
measuring a voltage Vm, with a voltage measuring instrument, between at least on further pair of the electrodes contacting the skin surface of the subject when the current source unit is passing the known current through the first pair of electrodes (404);
calibrating the measured voltage Vm with respect to the size of the subject to provide a calibrated voltage Vc (406A); and
calculating the impedance of the portion of the subject based on the calibrated voltage Vc and the known current (406B).

As described above, the size of the subject may be the size of the cross-section of the part of the body defined by the location of the electrodes. The size may be a circumference, a perimeter length, a thickness, a diameter, a radius, an axis length (e.g., cross sectional width or length), a volume, a surface area and a cross-sectional area.

For example, the calibrated voltage Vc may be defined as the voltage measured from the subject Vm that is calibrated to an expected voltage of a theoretical subject with the same biophysical profile with a standard chest perimeter length Pc, with the relationship between the perimeter length P and the voltage measured from a subject Vm being linear. As such, the calibrated voltage Vc may be determined according to the formula Vc=Vm−a(Pm−Pc) wherein a and Pc are constants.

It will be appreciated that the linear relationship between voltage readout and chest size is not limited to perimeter length. Other chest parameters, such as the thickness, the diameter, or an axis length such as cross-sectional width or cross-sectional length may have a linear relationship with the voltage readout as well. As such, the term P (i.e., the measured Pm and standard Pc) may alternatively refer to perimeter length, thickness, diameter, or an axis length such as crosssectional width or cross-sectional length.

The following description refers to the calibration methods.

The Calibration might be done in one of 2 ways:
1. Simplest—a formulation that correlates the relations between thorax width or other body parameter, and between the corrective formula is applied for all injections.
2. Fined tune: an optimized calibration function is applied for each injection separately.
In the linear case (will be described hereinafter), for example, for each injection, i, a coefficient aᵢ, bᵢ is assigned, and the calibration is then fully defined.

Electrodes Positioning Calibration

Conventional (Equi-Spaced) Electrodes Positioning:

EIT system typically requires equi-spaced skin surface electrodes. In an 8 electrodes example, as described in FIG. 12a, the electrodes distance is A, whereas A=(thorax perimeter)/8.

If the conventional positioning method will be applied for 2 subjects with thorax perimeter P₁ and P₂, the electrodes spacing will be A=P₁/8 and B=P₂/8 respectively, as can be seen in FIG. 12a. We can see that in this example, if subject 1 has bigger body dimensions than subject 2, the distance between the electrodes will also be bigger A>B, and proportional to the perimeter A/B=P₁/P₂

When electrodes are placed this way, for example on a chest belt with flexible distances, it is relatively simple to ensure equi-distances, but less practical to ensure a very accurate position of the electrodes on very specific points with regards to the body/anatomy. Due to this challenge, some of the EIT systems choose to limit the functionality to a functional-EIT (f-EIT) rather than taking a risk with inaccurate electrodes-anatomy positioning.

One object of the present invention is to provide a method of applying an EIT system with non-equi-spacing, using a compensating calibration.

According to said embodiment, the electrodes are not distributed in even distances. Instead, the some of the electrodes are placed by anatomical landmark.

Since the electrodes are placed by anatomical landmark, their position is better verified in comparison to equi-spacing method—and therefore a-EIT can be employed with lower risk of artefacts and ill posed solutions. The process can be schematically described:

Step 1: position electrodes, fixed position using anatomical landmarks;

Step 2: measure skin surface potential, V;

Step 3: calibrate skin surface potential, such that calibrated potential V_{c} is equal (or converge to) the theoretical skin potential that should have been derived using equi-spacing position; and,

Step 4: calculating map of conductivity or resistivity values.

The following provides detail on each of the above specified steps:

Step 1—Electrodes Position:

If we consider a 4 thorax electrodes example; with the equi-space method, the electrodes could be placed in equal distances, using a stretchable belt or otherwise, as described in FIG. 12b.

Example for anatomical based position:

2 electrodes under the right armpit and 2 electrodes under the left one.

For manufacturing convenience the electrodes in each pair are fixed distanced (see FIG. 12c).

Reference is now made to FIG. 12d illustrating the schematic electrodes position on the body of the above mentioned 4-electrodes example, for 2 patients with chest perimeters P₁ and P₂.

The following demonstrates the geometrical properties of this method:
The distance between left paired electrodes is pre-defined, and unrelated to the subject's specific dimensions (here is expressed as constant X), in contrast to the equi-spaced method where space would have been P/4.
   It should be noted that in the example described herein after, X is also identical to the distance between right paired electrodes though it is not a necessity.
While X₁=X₂=X, not all distances between electrodes are fixed. For example, if P₁>P₂ then Y₁>Y₂ (where Y stands for the distance between front-left and front-right electrodes).
   In contrast, with the equi-space method (FIG. 2) the distance between any 2 adjacent electrodes is identical A=A=A=A.
The proportion of distance between electrodes vary according to patient's dimensions. In this example, If P₁>P₂ then X/P₁<X/P₂. In contrast, with the equi-space method (FIG. 2) A/P₁=B/P₂

It should be emphasized that the number n of electrode in the system can be higher than 4; and the 4 electrode system described above is a sub-system of the n electrode system. In such a case, the calibration is performed only to the 4 electrode out of the n electrode system.

In other words, the 4 electrodes described above, can be a sub-system of n>4 electrodes. Similarly the injections in step 2 (will be described hereinafter) can be sub-set of a more complicated injections scheme.

Details for Step 2—Skin Surface Potential Measurement

The sequence of potential measurements will be done by the system definition (full EIT, parametric EIT, etc.) in the same sequence as if the positioning method was conventional.

In the example, suppose that for calculating EIT or pEIT conductivity values using 4 thorax electrodes setup, it will be required to sample 2 measurements of potential.

The 2 measurements are:

(V₁) current injection using electrodes 1,3
(V₂) current injection using electrodes 1,4.

Details for Step 3—Calibrating the Measured Potential

Calibrating the measured potential V, should compensate for the effect of the non-equi-spacing on the measured potential. Ideally the calibrated potential, should be identical to the potential that would have been measured as if the electrodes were equi-spaced.

In the example described above (e.g., in FIG. 4), a linear calibration is a good approximation to meets the above requirement. Linear calibration description:

For each measured potential Vₘ, for subject with thorax perimeter Pₘ, the calibrated potential V_{c} is calculated as follows:

In order to calibrate [symbol] , as if the perimeter Pₘ was , [symbol] , then: Vₘ=V_{c}+a(Pₘ−P_{c}) and therefore V_{c}=Vₘ−a(Pₘ−P_{c})

Selecting P_{c}=4× in our example, then the calibrated potential V_{c} represents the expected potential if an equi-spaced electrodes positioning was used.

Linear calibration is a specific example. Any calibration that will convert the measured potential Vₘ to the expected potential from equi-spaced positioning, would achieve the same goal.

Step 4: Calculating Map of Conductivity or Resistivity Values

Using the calibrated values V_{1c}, V_{c2}, . . . , V_{Nc} instead of the measured potential values V₁ₘ, V₂ₘ, . . . , V_{Nm}, no other change is needed in resistivity or conductivity calculation using an EIT or a pEIT solver for equi-spaced electrodes.

Delivery of Lower Frequency Alternating Current Calibration

The various options described for the bioimpedance measuring device 100 and its components as described with reference to FIGS. 1A-D above are also options for the discussion below regarding the frequency of the alternating current delivered to the subject.

As discussed above, the current source unit 230 may be an alternating current (AC) source capable of delivering a known alternating current in one or more defined amplitudes and frequencies. Any AC frequency may be used in the systems and methods disclosed herein.

Further, capacity effects on surface voltage/bioimpedance measurement of biological tissue are lower at lower AC frequencies and the total impedance is largely resistive. The capacity effects become negligible at sufficiently low AC frequencies, allowing the use of a quasi-static model of the biological volume conductor for impedance calculation. As such, the current source unit may be capable of generating a low frequency AC signal at a frequency of less than 100 kHz, less than 75 kHz, less than 50 kHz, less than 40 kHz, less than 30 kHz, less than 25 kHz, less than 20 kHz, less than 15 kHz, less than 10 kHz, less than 5 kHz, less than 2 kHz, less than 1 kHz, about 50 kHz, about 40 kHz, about 30 kHz, about 25 kHz, about 20 kHz, about 15 kHz, about 10 kHz, about 5 kHz, about 2, about 1 kHz, between 25 and 15 kHz, between 30 and 10 kHz and between 40 kHz and 5 kHz. In certain embodiments, the frequency of the AC signal is less than 20 kHz. In certain embodiments, the frequency of the low frequency AC signal is about 20 kHz.

Accordingly, with reference to FIG. 2, the present disclosure describes a method for measuring the bioimpedance of a portion of a subject, comprising the steps of:
passing a known current provided by a current source unit between a first pair of electrodes contacting the skin surface of the subject (402);
measuring a voltage Vm, with a voltage measuring instrument, between at least one further pair of the electrodes contacting the skin surface of the subject when the current source unit is passing the known current through the first pair of electrodes (404); and
calculating the bioimpedance of the portion of the subject based on the measured voltage Vm and the known current (406). As described above, the known current of the method may be an AC signal of any frequency.

Further, the known current may be a low frequency AC current at a frequency of less than 100 kHz, less than 75 kHz, less than 50 kHz, less than 40 kHz, less than 30 kHz, less than 25 kHz, less than 20 kHz, less than 15 kHz, less than 10 kHz, less than 5 kHz, less than 2 kHz, less than 1 kHz, about 50 kHz, about 40 kHz, about 30 kHz, about 25 kHz, about 20 kHz, about 15 kHz, about 10 kHz, about 5 kHz, about 2, about 1 kHz, between 25 and 15 kHz, between 30 and 10 kHz and between 40 kHz and 5 kHz. In certain embodiments, the frequency of the AC signal is less than 20 kHz. In certain embodiments, the frequency of the low frequency AC signal is about 20 kHz.

In addition, the devices and methods relating to delivery of low frequency alternating current may be used in the systems and methods disclosed herein in combination with body dimension calibration (as described below), breathing cycle calibration (as described below) and/or fixed resistive element calibration (as described below).

Breathing Cycle Calibration

The various options described for the bioimpedance measuring device 100 and its components as described with reference to FIGS. 1A-D above are also options for the breathing cycle calibration described below. Further, the devices and methods relating to breathing cycle calibration may be used in combination with the delivery of low frequency alternating current (as described above), body dimension calibration (as described above) and/or fixed resistive element calibration (as described below).

The voltage readout from a bioimpedance measuring device may also be affected by the breathing cycle of the subject, especially if the device is measuring chest bioimpedance. Chest impedance can change substantially depending on the portion of the chest volume taken up by air, which has a much greater resistivity than the surrounding tissue. The difference between the peaks and troughs in the voltage measured across the chest at different points in the inhalation/exhalation cycle can be about 30%. Therefore, controlling for this source of variability may improve the accuracy of bioimpedance measurements, especially chest bioimpedance measurements.

The bioimpedance measuring device of the disclosure, or the processor(s) therein involved in analyzing the voltage measurements, may be configured to calibrate the voltage measurements to the breathing cycle.

The device may control for the breathing cycle through a number of methods. Examples of such methods, inter alia, include the following:

Method 1: A series of voltage measurements may be taken over a period of time, encompassing in aggregate at least one full inhalation/exhalation cycle. The series of measurements may then be averaged, in order to reduce or eliminate the breathing artifact. In addition, the series of voltage measurements may be taken when the subject is breathing normally, i.e., engaging in “tidal volume breathing”, thus eliminating the added inaccuracies that may be introduced by irregular events such as deep breaths, shallow breaths, coughing, yawning and the like. Alternatively, the voltage measurements may be taken through a given period of time, then analyzed to select a portion where the changes in measured voltage is regular and thus represent a period of tidal volume breathing, and the subsequent analysis (e.g., averaging) limited to said portion. Further, if the dataset does not contain a qualified phase of a predetermined minimum number of tidal breathing cycles, the system can increase sampling time and/or provide notification. The predetermined minimum number of tidal cycles may be 2, 3, or more.

Method 2: Even during periods of irregular breathing, such as deep breathing or shallow breathing, the variability of voltage measured during exhalation peaks (a peak in exhalation corresponds to a lung volume trough, as well as a bioimpedance trough) is small compared the variability of voltage measured at other phases of the breathing cycle, such as the inhalation peak (a peak in inhalation corresponds to a lung volume peak, as well as a bioimpedance peak). A series of voltage measurements may be taken over a period of time, encompassing in aggregate at least two exhalation events. The measurement(s) at or near the voltage minimums (troughs), representing the peak(s) of exhalation, may be selectively used in subsequent averaging and analysis. In addition, the measurements taken may be limited to when the subject is breathing normally, i.e., engaging in “tidal breathing”, thus eliminating the added inaccuracies that may be introduced by irregular events such as deep breaths, shallow breaths, coughing, yawning and the like. Alternatively, limiting analysis to the voltage measurements made at the exhalation peaks may obviate the need to select voltage measurement acquisition to periods of tidal breathing.

Accordingly, with reference to FIG. 4A, the present disclosure describes a method for measuring the impedance of a portion of a subject, comprising the steps of:
passing a known current provided by a current source unit between a first pair of electrodes contacting the skin surface of the subject (402);
measuring a voltage Vm, with a voltage measuring instrument, between at least one further pair of the electrodes contacting the skin surface of the subject when the current source unit is passing the known current through the first pair of electrodes (404); and
calibrating the measured voltage Vm with respect the breathing cycle of the subject (406A′); and
calculating the impedance of the portion of the subject based on the calibrated voltage V, and the known current (406B).

With reference to FIG. 4B, the step of breathing cycle calibration (406A′) may be performed according to an algorithm comprising the steps of:
taking a plurality of voltage measurements over a period of time encompassing in aggregate at least one full inhalation/exhalation cycle (502); and
averaging said plurality of voltage measurements (504).

Alternatively, with reference to FIG. 4C, the step of breathing cycle calibration (406A′) may be performed according to an algorithm comprising the steps of:
taking a first plurality of voltage measurements over a period of time encompassing in aggregate at least two exhalation events (602);
from said first plurality of voltage measurements, selecting a second plurality of voltage measurements at or near the voltage troughs (604);
averaging said second plurality of voltage measurements (606).

Fixed Resistive Element Calibration

The various options described for the bioimpedance measuring device 100 and its components as described with reference to FIGS. 1A-D above are also options for the devices and methods relating to fixed resistive element calibration described below. Further, fixed resistive element calibration may be used in combination with the delivery of low frequency alternating current (as described above), body dimension calibration (as described above) and/or breathing cycle calibration (as described above).

Bioimpedance measurements can influenced by factors such as (a) variability in temperature; (b) variability of electrical components, e.g., between equivalent but not identical components used on different devices, in the form of voltage drift, bias current and the like; and (c) the tolerances of the components, e.g., the variability of circuit properties within different units of the same model with the same components. As such, there is a desire to mitigate against such variability to achieve higher precision in the voltage measurements, while avoiding high cost. Further, there is a need to derive the absolute impedance from the impedance calculated from the known current being injected, together with the measured voltage.

Referring now to FIGS. 5A-B, the bioimpedance measuring device 100 of the disclosure (as shown in, e.g., FIGS. 1A and 1B), which includes the controller 225 having the current source unit 230, the voltage measuring unit 240, at least one microprocessor 250, and optionally the multiplexor 260, may further include a fixed resistive element 350 that is connectable to the current source unit 230 and the voltage measurement unit 240. The fixed resistive element 350 may have known and stable resistance R, which is unaffected (or minimally affected) by time and environmental factors, e.g., temperature, humidity, presence of electromagnetic fields, and the like. The controller 225 may be configured to calculate a system impedance SI, reflecting the circuit elements of the components comprising the bioimpedance measuring device 100, together with the sources of error that the components may introduce, by injecting a known current through the fixed resistive element 350 and measuring the resulting voltage. That is, the controller may be configured to calculate a system impedance SI based on the voltage measured during the injection of a known current through the fixed resistive element 350. The bioimpedance measurements obtained from the electrodes 130 contacting the subject may then be calibrated with respect to said system impedance SI.

A bioimpedance measurement session (i.e., through the measurement of voltages through electrodes connected to the subject 10 during AC injection) to obtain a measured bioimpedance BIM may be accompanied (before or after) by the determination of the impedance through the fixed resistive element 350 to obtain the system impedance SI. The measured bioimpedance BIM may be calibrated against the system impedance SI and the known resistance R of the fixed resistive element 350 to calculate the calibrated bioimpedance BIC according to the formula:

$$\frac{B I M}{S I} = \frac{B I C}{R}$$

such that the calibrated bioimpedance BIM may be expressed as:

$$B I C = \frac{B I M}{S I} \cdot R$$

The calibrated bioimpedance BIC is robust in being resistant to many potential sources of variation, for example, as describe above: (a) variability in temperature; (b) variability of electrical components, e.g., between equivalent but not identical components used on different devices, in the form of voltage drift, bias current and the like; and (c) the tolerances of the components, e.g., the variability of circuit properties within different units of the same model with the same components.

The following is an Illustrative Example:

The resistance of the fixed resistive element 350 is known to be 10Ω, the measured bioimpedance BIM (through the electrodes 130 contacting the subject) is measured as 2500 (arbitrary units), and the system impedance SI obtained from connecting the fixed resistive element 400 to the current source unit 230 and the voltage measurement unit 240 is measured as 1000 (arbitrary unites).

Based on these suggested parameters (BIM=2500 arbitrary units, SI=1000 arbitrary units and R=10Ω) the calibrated bioimpedance BIC in calculated to be 25Ω according to the formula:

$$\frac{2500}{1000} \cdot 10 Ω = 25 Ω$$

If, for example, the current source unit 230 function is inconsistent (over time or over different devices), and in a separate instance, produces an alternating current that is 5% weaker, the calibrated bioimpedance is unaffected. The calibrated bioimpedance BIC will remain the same because the measured bioimpedance BIM and the system impedance SI will be similarly affected, thus canceling each other out. Based on the current source unit 230 being 5% weaker, the measured bioimpedance BIM may be 5% lower, say 2375 arbitrary units, and the system impedance SI may also be 5% lower, say 950 arbitrary units. The known resistance R of the fixed resistive element 350 remains 10Ω. As such, the calibrated bioimpedance will still be calculated to the same 25Ω regardless of the variations present in the current course unit 230, according to the formula:

$$\frac{2375}{950} \cdot 10 Ω = 25 Ω$$

Accordingly, with reference to FIG. 6, the present disclosure describes a method for measuring the impedance of a portion of a subject, comprising the steps of:
passing a known current provided by a current source unit between a first pair of electrodes contacting the skin surface of the subject (1402);
measuring a first voltage, with a voltage measuring instrument, between at least one further pair of the electrodes contacting the skin surface of the subject when the current source unit is passing the known current through the first pair of electrodes (1404);
calculating the measured bioimpedance BIM of the portion of the subject based on the first voltage and the known current (1406);
passing the known current provided by the current source unit through a fixed resistive element having a resistance R (1408);
measuring a second voltage, with the voltage measuring instrument, when the current source unit is passing the known current through the fixed resistive element (1410);
calculate a system impedance SI based on the second voltage and the known current (1412); and
calibrating the measured bioimpedance BIM with respect to the system impedance SI to derive the calibrated bioimpedance BIC (1414). As described above, the calibrated bioimpedance BIC may be calculated according to the formula:

$$B I C = \frac{B I M}{S I} \cdot R$$

### EXAMPLES

Examples are given in order to prove the embodiments claimed in the present invention. The example, which is a clinical test, describes the manner and process of the present invention and set forth the best mode contemplated by the inventors for carrying out the invention, but are not to be construed as limiting the invention.

### Example 1

Methods

We simulated a portable bio-impedance system that consists of four electrodes. The system employs a parametric EIT algorithm to reconstruct the resistivity values of each lung from two impedance measurements. In each measurement, the voltage between the one electrode pair was measured while current was injected through a second pair of electrodes. A second order modified Newton-Raphson algorithm was used to calculate the optimal values for the two parameters, i.e., the resistivity of the two lungs. The reconstruction algorithm (i.e., the Newton-Raphson algorithm) was based on a predefined and known fixed thoracic geometry with a perimeter of ˜100 cm. For such a system to correctly measure and monitor lung edema in subjects of other thoracic sizes (i.e., a lung perimeter of less than 100 cm or greater than 100 cm), it is important to validate a calibration curve for adjusting the physical voltage measurements made on the various subjects to a calculated expected value of a hypothetical patient having a chest perimeter of 100 cm.

The surface potentials were calculated by solving the following governing Laplace equation with Neumann type boundary condition, which is an extension of Ohm's law:

$$V \cdot ( \frac{1}{ρ} V φ ) = 0$$

$$\frac{1}{ρ} \frac{\partial φ}{\partial n} = \{ \begin{matrix}J , & on electrode position \\ 0 , & elsewhere\end{matrix}$$

where ρ (Ωcm) is the tissue resistivity, φ (V) the electrical potential, J (A m⁻²) the injected current density and n is a unit vector normal to the boundary. The boundary condition specifies that no current flows into the surrounding insulating air except at the locations of the injecting electrodes. In the physical model, several assumptions and simplifications were applied, including the quasi-static approximation and linearity and isotropy of the biological volume conductor. The finite-volume method was employed for the discretization and the numerical solution of the integral presentation of the governing equation by taking a surface integral on the PDE and applying Gauss's divergence theorem:

$${∮}_{S} \frac{1}{ρ} V φ \cdot d s = 0$$

where ds (cm⁻²) is a vector surface element.

A two-dimensional thorax model that is based on an axial CT image was employed for all simulations. Referring now to FIG. 7A, the original image was segmented into 4 tissue types, each of which was given an appropriate resistivity value for a 20 kHz electrical field from the literature (e.g., Gabriel S, Lau R W, Gabriel C, Phys Med Biol 1996: 2231-93): ρₕₑₐᵣₜ=143Ω, ρ_{soft tissue}=300 Ωcm, ρ_{bone}=5000 Ωcm. The lungs were assigned with varying values ranging from 500 to 1500 Ωcm representing wet and dry lungs, respectively (with nonpathological values around 1000 Ωcm). As shown in FIG. 7B, the image was then sampled into a lower resolution of 20×20 pixels, with a spatial resolution ranging between Δh=1 to 2 cm, corresponding to geometry perimeters between 66 and 132 cm, respectively.

Referring now to FIG. 8, electrodes were positioned on the two sides of the geometry in 2 sets of 2 electrodes. Electrodes 130A and 130B were positioned +4.5 and −4.5 cm along the surface relative to an angular position of 0°, so that the surface distance between them was kept at 9 cm.

Similarly, electrodes 130C and 130D were positioned +4.5 and −4.5 cm along the surface relative to an angular position of 180°, so that the surface distance between them was kept at 9 cm as well.

The first measurement was defined by injecting current between electrodes 130A and 130C, and sampling the voltage between electrodes 130B and 130D, Vᵢₙⱼ₁ The second measurement was defined by injecting current between electrodes 130A and 130D, and sampling the voltage between electrodes 130B and 130C, Vᵢₙⱼ₂.

Results

Bilateral edema was first simulated by setting both lung resistivity values to either 500, 1000 or 1500 Ωcm. The two voltage measurements, Vᵢₙⱼ₁ and Vᵢₙⱼ₂ were calculated for a range of thoracic perimeters. For each measurement, Vᵢₙⱼ₁ and Vᵢₙⱼ₂ the voltage calculated for each perimeter length was divided by the voltage measurement calculated for a perimeter length of 100 cm to obtain the relative voltage values (i.e., relative to the 100 cm perimeter length).

Referring now to FIGS. 9A-C, the relative voltage values were compared to those obtained for a perimeter of 100 cm. For all three values of lung resistivity, 500 Ωcm (FIG. 9A), 1000 Ωcm (FIG. 9B), and 1500 Ωcm, (FIG. 9C), a linear relationship was found between the relative measurement and the perimeter (linear regression, R²20>0.98, Tables 1 and 2).

Essentially the same linear relationship was found with both voltage measurements Vᵢₙⱼ₁ and Vᵢₙⱼ₂.

TABLE 1
Linear fit parameters for injection 1. Line equation:
Vᵢₙⱼ₁ = a · perimeter − b

| ρ_{lung1} | ρ_{lung2} | a | b |
|---|---|---|---|
| 500 | 500 | 0.01686 | −0.7397 |
| 1000 | 1000 | 0.01725 | −0.7860 |
| 1500 | 1500 | 0.01724 | −0.7877 |
| 500 | 1200 | 0.01653 | −0.6963 |
| 1200 | 500 | 0.01763 | −0.8394 |
| | mean | 0.0171 ± 0.0004 | −0.7698 ± 0.0542 |

TABLE 2
Linear fit parameters for injection 2. Line equation:
Vᵢₙⱼ₂ = a · perimeter − b

| ρ_{lung1} | ρ_{lung2} | a | b |
|---|---|---|---|
| 500 | 500 | 0.01577 | −0.6192 |
| 1000 | 1000 | 0.01607 | −0.6547 |
| 1500 | 1500 | 0.01606 | −0.6559 |
| 500 | 1200 | 0.01560 | −0.5933 |
| 1200 | 500 | 0.01642 | −0.7043 |
| | mean | 0.0160 ± 0.0003 | −0.6455 ± 0.0420 |

Referring now to FIGS. 10A-B, unilateral edema was simulated by setting the left lung resistivity to 500 Ωcm and the right lung resistivity to 1200 Ωcm (FIG. 10A), and then switching the resistivity values between the lungs (FIG. 10B). The two voltage measurements, Vᵢₙⱼ₁ and Vᵢₙⱼ₂ were calculated for a range of thoracic perimeters, and the relative values compared to those obtained for a perimeter of 100 cm. Again, a linear relationship was found between the relative measurement and the perimeter (linear regression, R²>0.98, Tables 1 and 2).

The average linear fits for injections 1 and 2, incorporating the three modes of bilateral edema and the two modes of unilateral edema tested and described above (in FIGS. 9A-10B and Tables 1-2),were:

Vᵢₙⱼ₁=0.017*perimeter−0.7698

Vᵢₙⱼ₂=0.0160*perimeter−0.6455

Thus, voltage measurement calibration for varying thoracic perimeters can be established via simple linear curves. Both measurements show very similar calibration curves, and in practice, one curve can be used to fit all data. This conclusion is valid for both bilateral and unilateral pulmonary edema.

### Example 2

With reference to FIGS. 11A-E, we connected four electrodes to a subject and conducted a continuous (fast sampling) bioimpedance monitoring for over 10 seconds. Two pairs of electrodes, an injecting pair and a measuring pair, were placed on the thorax. Each pair contained one electrode that was placed on the left side of the thorax and another electrode that was placed on the right side of the thorax. The voltage resulting from the injected current was monitored during a variety of non-tidal breathing modes: normal breathing (FIGS. 11A-B), deep breathing (FIG. 11C), no breathing (FIG. 11D) and deep inhalation followed by complete emptying of the lungs (FIG. 11E).

We found that the difference in breathing inhale/exhale voltage peaks is significant, ˜30%, confirming that the breathing artifact is a major source of noise. We also found that the heart cycle was not a significant source of noise.

In addition, we found that the variability of the voltage measured at different phases of the breathing cycle was not uniform. The variability of air intake by the lungs is largely controlled by increasing or decreasing the level of lung expansion during inhalation while the level of lung contraction during exhalation remains relatively constant. During both normal breathing and deep breathing, the variability among individual voltage peaks (corresponding to peak inhalation) was higher than the variability among the individual voltage troughs corresponding to peak exhalation (see FIGS. 11A and 11C). In addition, the difference in the measured voltage during deep breathing as compared to normal breathing is primarily reflected in the change in the voltage peaks (corresponding to peak inhalation), with the change in the voltage troughs (corresponding to peak exhalation) being substantially smaller. See FIGS. 11B and 11C.

As such, the breathing artifact of bioimpedance measurements of the chest may be mitigated or eliminated by limiting the analysis of the measured voltage to those measured at or near the voltage troughs, corresponding to the exhalation peaks.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A method for measuring the impedance of a portion of a subject:
a. passing a known current provided by a current source unit between a first pair of electrodes contacting the skin surface of said subject;
b. measuring a voltage [symbol] with a voltage measuring instrument, between at least one second pair of electrodes contacting said skin surface of said subject when said current source unit is passing said known current through said first pair of electrodes;
c. calculating the bio-impedance of said portion of said subject based on said known current and the calibrated voltage [symbol] ;
wherein said calibrated voltage [symbol] is calculated according to the following calibration formula:
V_{c}=Vₘ−( [symbol] _{A}_{ₘ}[(J(□)]\vec{r}′(\vec{r}))α(r)d\vec{r}− [symbol] _{A}_{_{c}}[(J(□)]\vec{r}′(\vec{r}))α(r)d\vec{ [symbol]})
wherein Aₘ is the volume of said portion of said subject enclosed by said second pair of electrodes; A_{c} is the volume of a standard portion of a measured subject enclosed by said second pair of electrodes; α(r) is a function containing the resistivity of a body according to the radius the cross section of said subject; and J(\vec{r}(\vec{r})) is the Jacobian of coordinate transformation from Cartesian coordinates \vec{r}′ to elliptic coordinates \vec{r}:
$$J ( {{r}^{′}}^{} ( {r}^{->} ) ) = Det \vert \frac{d {r}^{->} ( {r}^{->} )}{d {r}^{->}} \vert$$
further wherein said α(r) is calculated according to a solution of the poission equation:
$$v \cdot ( \frac{1}{ρ} ∇ ϕ ) = - {l}_{y} ;$$
said φ is the electric potential according as a function of position on the thorax of said subject; said ρ is the impedance as a function of position in said thorax; and l_{γ} is zero except on the surface of said thorax.

2. The method according to claim 1, wherein at least one of the following is being held true (a) said cross section is an elliptic cross-section; (b) said known current is an alternating current having a frequency of 20 kHz or less; (c) said controller is configured to calculate the bio-impedance of said portion of said subject based on said calibrated voltage v_{c}, and said known current; (d) said known current is an alternating current having a frequency of 40 kHz or less; (e) said known current is an alternating current having a frequency of 60 kHz or less; and any combination thereof

3. The method according to claim 1, wherein said step of calculating said impedance comprises sub-step of:
calculating the bio-impedance of said portion of said subject based on said calibrated voltage v_{c} and said known current.

4. The method according to claim 1, wherein, when a(r) is a constant function, said calibration formula is the following linear formula: v_{c}=vₘ−B(Pₘ−P_{c}), where B is a constant, Pₘ is the measured cross-section, and P_{c} is the standard cross-section size;
further wherein B is deduced by linear regression based on an empiric measurement of thorax width and cross thorax impedance.

5. The method of claim 1, wherein said step of calculating the bio-impedance comprises substeps of:
calibrating said measured voltage vₘ with respect to the breathing cycle of said subject to provide a calibrated voltage v_{c}; and
calculating the bio-impedance of said portion of said subject based on said calibrated voltage v_{c}, and said known current.

6. The method according to claim 5, wherein said step of breathing cycle calibration is performed according to an algorithm comprising further steps of:
taking a first plurality of voltage measurement over a period of time encompassing in aggregate at least two exhalation events;
from said first plurality of voltage measurements, selecting a second plurality of voltage measurements at or near the voltage troughs;
averaging said second plurality of voltage measurements.

7. The method according to claim 1, wherein said vₘ is calibrated in respect to skin potential that should have been derived using either equi-spacing position, non-equi-spacing; and any combination thereof.

8. A device configured to measure the impedance of a portion of a subject, comprising:
a current source unit capable of passing a known current through said subject's chest through a first pair of electrodes;
a voltage measuring unit capable of measuring a voltage between at least one second pair of electrodes when said current source unit is passing said known current through said portion of said subject through said first pair of electrodes, and when said first and said second pair of electrodes are placed on said subject; and
a controller, comprising at least one processor, configured to determine the impedance of said portion of said subject based on said known current and calibrated voltage [symbol] value based upon measured voltage; said calibrated voltage [symbol] is calculated by the formula:
V_{c}=Vₘ−( [symbol] _{A}_{ₘ}[(J(□)]\vec{r}′(\vec{r}))α(r)d\vec{r}− [symbol] _{A}_{_{c}}([J(□)]\vec{r}′(\vec{r}))α(r)d\vec{ [symbol]})
where Aₘ is the volume of a portion of said subject enclosed by said second pair of electrodes; A_{c} is the volume of a standard portion of a measured subject enclosed by said second pair of electrodes; α(r) is a function containing the resistivity of a body according to the radius of a cross section of said subject; and J(\vec{r}′(\vec{r})) is the Jacobian of coordinate transformation from Cartesian coordinates \vec{r}′ to elliptic coordinates \vec{r}:
$$J ( {{r}^{′}}^{} ( {r}^{->} ) ) = Det \vert \frac{d {{r}^{′}}^{} ( {r}^{->} )}{d {r}^{->}} \vert$$
further wherein said α(r) is calculated according to a solution of the poission equation:
$$∇ \cdot ( \frac{1}{ρ} ∇ ϕ ) = - {l}_{g} ;$$
said φ is the electric potential according as a function of position on the thorax of said subject; said ρ is the impedance as a function of position in said thorax; and l_{γ} is zero except on the surface of said thorax.

9. The device according to claim 8, wherein at least one of the following is being held true (a) said cross section is an elliptic cross section; (b) said known current is an alternating current having a frequency of 20 kHz or less; (c) said known current is an alternating current having a frequency of 40 kHz or less; (d) said known current is an alternating current having a frequency of 60 kHz or less; and any combination thereof.

10. The device according to claim 8, wherein is constant, said calibration formula is the Following linear formula: v_{c}=vₘ−B(Pₘ−P_{c}), where B is a constant, Pₘ is the measured cross-section, and P_{c} is the standard cross-section size.

11. The device according to claim 10, wherein B is deduced by linear regression based on an empiric measurement of thorax width and cross thorax impedance.

12. The device claim 8, wherein at least one of the following is being held true (a) said controller is configured to calibrate the measured voltage vₘ with respect to the breathing cycle of said subject to provide a calibrated voltage v_{c} (b) said controller is configured to calculate the bio-impedance of said portion of said subject based on said calibrated voltage v_{c}, and said known current; and any combination thereof.

13. v_{c} The device according to claim 8, wherein said breathing cycle calibration is performed according to an algorithm comprising steps of:
taking a plurality of voltage measurements over a period of time encompassing in aggregate at least one full inhalation/exhalation cycle; and,
evaraging said plurality of voltage parameters.

14. The device according to claim 8, wherein the breathing cycle calibration is performed according to an algorithm comprising steps of:
taking a first plurality of voltage measurement over a period of time encompassing in aggregate at least two exhalation events;
from said first plurality of voltage measurements, selecting a second plurality of voltage measurements at or near the voltage troughs;
averaging said second plurality of voltage measurements.

15. The device according to claim 8, wherein said vₘ is calibrated in respect to skin potential that should have been derived using either equi-spacing position, non-equi-spacing; and any combination thereof.

16. The device according to claim 8, further comprising a fixed resistive element having a having a resistance R connectable to said current source unit and said voltage measuring unit, wherein said controller is configured to calculate a system impedance SI based on the voltage measured during the injection of a known current through the fixed resistive element, as well as to calibrate the measured bioimpedance BIM with respect to the system impedance SI to obtain a calibrated bioimpedance BIC.

17. The device according to claim 16, wherein the calibrated bioimpedance BIC is calculated according to the formula: BIC=(BIM/SI)R.

18. The device according to claim 8, wherein said device is configured to perform at least one process selected from the group consisting of plethysmograpy, impedance cardiography, pneumography, organ volumetry, tissue volumetry, tissue characterization, edema detection, ischemia detection, graft viability monitoring and graft rejection monitoring.

19. The device according to claim 18, wherein the tissue characterization is cancer detection.

20. The device according to claim 8, wherein at least one of the following is being held true (a) said device is configured to measure impedance in the chest of said subject; (b) said device is incorporated into an electrical impedance tomography (EIT) system; (c) wherein said device is incorporated into a parametric electrical impedance tomography (EIT) system; (d) wherein said device is configured to measure the level of pulmonary edema in at least one lung of said subject; and any combination thereof.
